(19) United States
(12) Patent Application Publication
(10) Pub. No.: US 2025/0118400 A1
(43) Pub. Date: Apr. 10, 2025

(54) MEDICAL INFORMATION PROCESSING APPARATUS, MEDICAL INFORMATION PROCESSING SYSTEM AND MEDICAL INFORMATION PROCESSING METHOD

(71) Applicant: CANON MEDICAL SYSTEMS CORPORATION, Tochigi (JP)

(72) Inventors: Kanako AIZAWA, Yaita (JP); Kohei WATANABE, Utsunomiya (JP); Mami TAKAHASHI, Nasushiobara (JP); Mayumi SAKAGAMI, Ibaraki (JP); Tomoki FUJITO, Nasushiobara (JP); Satoru KOMATSU, Melrose, MA (US)

(73) Assignee: CANON MEDICAL SYSTEMS CORPORATION, Tochigi (JP)

(21) Appl. No.: 18/909,810

(22) Filed: Oct. 8, 2024

Related U.S. Application Data
(60) Provisional application No. 63/589,185, filed on Oct. 10, 2023.

(51) Int. Cl. G16H 10/60; G16H 30/40

(52) U.S. Cl. CPC G16H 10/60; G16H 30/40

(57) ABSTRACT

A medical information processing apparatus according to an embodiment includes processing circuitry configured to acquire examination data including examination values of biological examinations performed on the same subject at a plurality of time points; create a map based on the examination data, the map including a time-series element group in which a plurality of elements corresponding to the biological examinations performed at the plurality of time points and having a visual representation according to a degree of risk are arranged in a time-series order along a first direction; and control a display unit to display the map.

### CROSS REFERENCE TO RELATED APPLICATION

This application is based upon and claims the benefit of priority from the U.S. Provisional Application No. 63/589,185, filed on Oct. 10, 2023, the entire contents of which are incorporated herein by reference.

### FIELD

Embodiments disclosed in the present specification and drawings relate to a medical information processing apparatus, a medical information processing system, and a medical information processing method.

### BACKGROUND

An examination (biological examination) using a biomarker is performed as an indicator of a change in disease state or a therapeutic effect. The biomarker refers to a substance such as a protein measured in a body fluid such as blood, and the presence or progress of a disease is reflected in its concentration. For example, the biomarker is a protein, a gene, or the like whose blood concentration changes, reflecting the presence or progress of various cancers such as breast cancer and colorectal cancer.

In recent years, a liquid biopsy (LB) for diagnosing a lesion, predicting a therapeutic effect, and the like using a body fluid such as blood or urine has been known. The liquid biopsy is a technique for detecting a disease such as cancer at an early stage or measuring a therapeutic effect by a drug or the like by detecting a specific protein from blood or the like and using the detected specific protein as a biomarker. The liquid biopsy has an advantage that it can be repeated without placing a burden on a body as compared to a tissue biopsy.

A relationship between a magnitude of an examination value and a degree of a risk level varies depending on the type of biomarker. That is, for one biomarker, the risk increases as the examination value increases, and for another biomarker, the risk increases as the examination value decreases. Therefore, it is not easy to grasp the risk level only by confirming at the examination value for the biomarker.

### BRIEF DESCRIPTION OF THE DRAWINGS

FIG. 1 is a view illustrating an example of a configuration of a medical information processing system according to an embodiment;

FIG. 2 is a block diagram illustrating an example of a configuration of a medical information processing apparatus according to a first embodiment;

FIG. 3 is a block diagram illustrating an example of a configuration of a medical information storage apparatus according to the first embodiment;

FIG. 4 is a view illustrating an example of a patient information DB according to an embodiment;

FIG. 5 is a view illustrating an example of a biomarker DB according to an embodiment;

FIG. 6 is a view illustrating an example of a medical image DB according to an embodiment;

FIG. 7 is a view illustrating an example of the order of biomarker examination and imaging (mammography examination);

FIG. 8 is a flowchart for explaining an example of a method of creating a heat map;

FIG. 9 is a view for explaining a risk level of an examination value for each biomarker;

FIG. 10 is a view illustrating an example of a heat map according to an embodiment;

FIG. 11 is a view illustrating an example of an output screen of a medical information processing apparatus according to a first embodiment;

FIG. 12 is a view illustrating an example of the output screen of the medical information processing apparatus according to the first embodiment;

FIG. 13 is a view illustrating an example of the output screen of the medical information processing apparatus according to the first embodiment;

FIG. 14 is a view illustrating an example of the output screen of the medical information processing apparatus according to the first embodiment;

FIG. 15 is a view illustrating an example of the output screen of the medical information processing apparatus according to the first embodiment;

FIG. 16 is a view illustrating an example of the output screen of the medical information processing apparatus according to the first embodiment;

FIG. 17 is a view illustrating an example of the output screen of the medical information processing apparatus according to the first embodiment;

FIG. 18 is a view illustrating an example of the output screen of the medical information processing apparatus according to the first embodiment;

FIG. 19 is a view illustrating an example of the output screen of the medical information processing apparatus according to the first embodiment;

FIG. 20 is a view illustrating an example of the output screen of the medical information processing apparatus according to the first embodiment;

FIG. 21 is a view illustrating an example of the output screen of the medical information processing apparatus according to the first embodiment;

FIG. 22 is a block diagram illustrating an example of a configuration of a medical information processing apparatus according to a second embodiment;

FIG. 23 is a view illustrating an example of an output screen of the medical information processing apparatus according to the second embodiment;

FIG. 24 is a view illustrating an example of the output screen of the medical information processing apparatus according to the second embodiment; and

FIG. 25 is a view illustrating an example of the output screen of the medical information processing apparatus according to the second embodiment.

### DETAILED DESCRIPTION

Hereinafter, embodiments of a medical information processing apparatus, a medical information processing system, and a medical information processing method will be described with reference to the drawings. Note that, in the following description, components having substantially the same functions and configurations are denoted by the same reference numerals, and redundant description will be given only when necessary.

### <Medical Information Processing System>

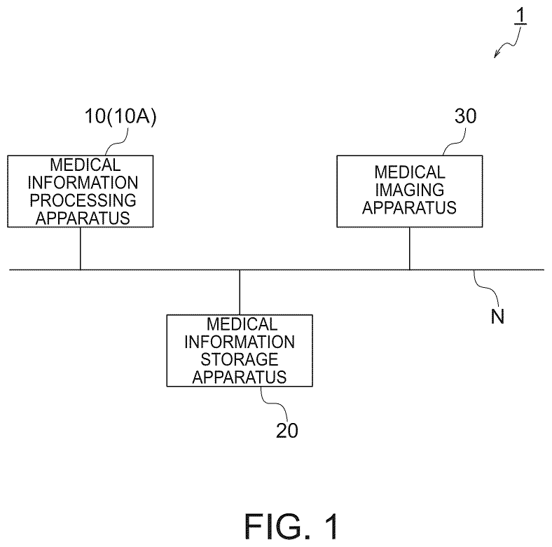

A medical information processing system 1 common to each embodiment will be described with reference to FIG. 1. The medical information processing system 1 includes a medical information processing apparatus 10 (10A), a medical information storage apparatus 20, and a medical imaging apparatus 30. The respective apparatuses are communicably connected by a communication network N. A configuration and a protocol of the communication network N are not particularly limited. Note that the communication network N may be an in-hospital network or may include a public network such as the Internet.

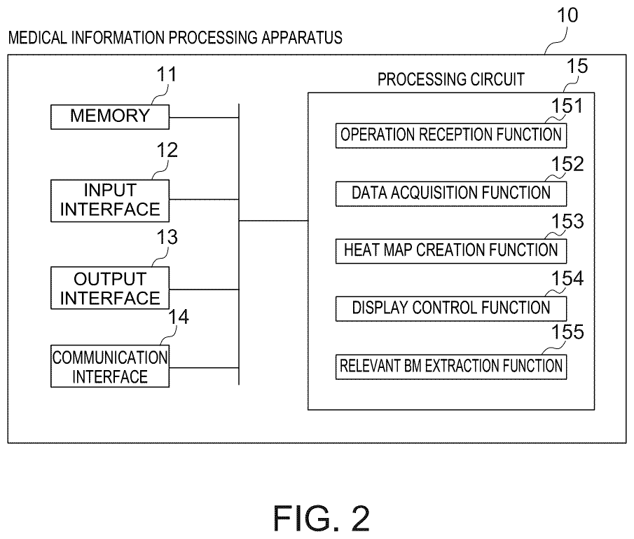

The medical information processing apparatus 10 is configured to display results of a plurality of examinations performed on an examination subject (hereinafter, also simply referred to as a “subject”) such as a patient. Details of the medical information processing apparatus 10 will be described below with reference to FIG. 2.

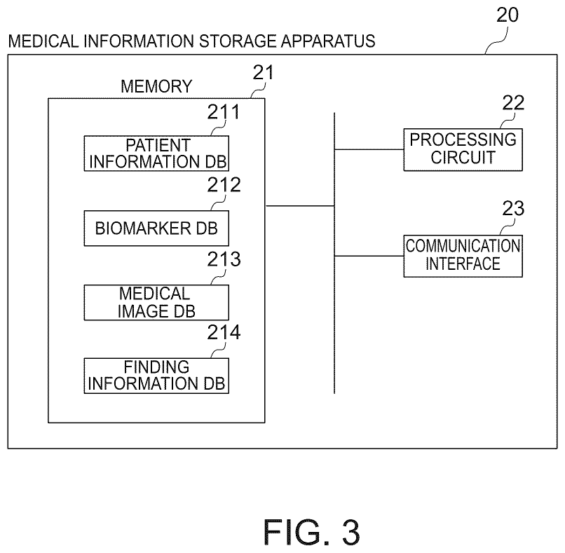

The medical information storage apparatus 20 stores examination data including examination values of biological examinations performed on the same subject at a plurality of time points. Although described in detail below with reference to FIG. 3, the medical information storage apparatus 20 is configured to store information on an examination subject (patient information), a medical image captured by the medical imaging apparatus 30, and an examination value for a biomarker examination (liquid biopsy examination or the like). The medical image is, for example, image data obtained from a mammography apparatus, an X-ray computed tomography (CT) apparatus, a magnetic resonance imaging (MRI) apparatus, or the like.

Upon receiving a request from the medical information processing apparatus 10, the medical information storage apparatus 20 provides the stored information to the medical information processing apparatus 10.

The medical imaging apparatus 30 is an apparatus that performs an examination by imaging an examination subject, and is, for example, a mammography apparatus. Note that the medical imaging apparatus 30 may be another imaging apparatus such as an X-ray CT apparatus, an MRI apparatus, or an ultrasonic diagnostic apparatus.

### Medical Information Processing Apparatus (First Embodiment)

A medical information processing apparatus 10 according to a first embodiment will be described in detail with reference to FIG. 2.

As illustrated in FIG. 2, the medical information processing apparatus 10 includes a memory 11, an input interface 12, an output interface 13, a communication interface 14, and a processing circuit 15. Hereinafter, details of each configuration will be described.

The memory 11 is connected to the processing circuit 15 and stores various types of information used in the processing circuit 15. The memory 11 is realized by, for example, a random access memory (RAM), a semiconductor memory element such as a flash memory, a hard disk, an optical disk, or the like. The memory 11 stores various programs necessary for the processing circuit 15 to execute each function, various data processed by the programs, and the like. Note that various data handled in the present specification are typically digital data.

The input interface 12 receives various operations from a user such as a doctor, converts the received operations into electrical signals, and outputs the electrical signals to the processing circuit 15. The input interface 12 is realized by, for example, a mouse, a keyboard, a touch panel, a trackball, a manual switch, a foot switch, a button, a joystick, or the like.

The output interface 13 presents medical information such as various examination results to the user of the medical information processing apparatus 10. The output interface 13 includes a display unit such as a liquid crystal display, and displays information such as an image. Specifically, the output interface 13 displays an examination value of a liquid biopsy examination (LB examination), a medical image such as mammography, or a graphical user interface (GUI) that receives a user's operation. Note that the output interface 13 may include a voice output unit (speaker) and may output information to be presented to the user by voice.

The communication interface 14 communicates with other apparatuses (such as the medical information storage apparatus 20) via a communication network in accordance with various communication protocols.

The processing circuit 15 is an arithmetic circuit that performs various arithmetic operations, and controls the operation of the medical information processing apparatus 10. The processing circuit 15 has an operation reception function 151, a data acquisition function 152, a heat map creation function 153, a display control function 154, and a relevant BM extraction function 155. The operation reception function 151 is an example of an operation reception unit, the data acquisition function 152 is an example of an acquisition unit, the heat map creation function 153 is an example of a map creation unit, the display control function 154 is an example of a display control unit, and the relevant BM extraction function 155 is an example of an extraction unit.

In the present embodiment, each processing function executed by each of the operation reception function 151, the data acquisition function 152, the heat map creation function 153, the display control function 154, and the relevant BM extraction function 155 is stored in the memory 11 in the form of a computer-executable program. The processing circuit 15 includes a processor, reads programs from the memory 11, and execute the programs, such that a function corresponding to each program is realized. In other words, the processing circuit 15 in a state of reading each program has each function illustrated in the processing circuit 15 of FIG. 2.

Note that, in FIG. 2, although a case where a processing function of each of the operation reception function 151, the data acquisition function 152, the heat map creation function 153, the display control function 154, and the relevant BM extraction function 155 is realized by a single processing circuit 15 is described, an embodiment is not limited thereto. For example, the processing circuit 15 may be configured by combining a plurality of independent processors, and each processor may execute each program to realize each processing function. Each processing function of the processing circuit 15 may be realized by being appropriately distributed or integrated into a single or a plurality of processing circuits.

Next, details of each processing function of the processing circuit 15 will be described.

The operation reception function 151 receives an operation input by a user (a doctor or the like) of the medical information processing apparatus 10 via the input interface 12. Specifically, an operation on a graphical user interface (GUI) (a button, a pull-down menu, a tab, or the like) displayed on the display unit of the output interface 13 is received. For example, the operation reception function 151 receives a selection operation for one or a plurality of elements of the heat map displayed on the output interface 13.

The data acquisition function 152 acquires an examination result (examination value) of a biological examination such as an LB examination. The data acquisition function 152 acquires the examination value of the patient designated by the user from the medical information storage apparatus 20. Here, the “biological examination” is an examination using a biomarker (biomarker examination). Note that the biomarker examination may be an examination by a liquid biopsy (LB examination).

The examination values acquired by data acquisition function 152 may be examination results of biological examinations performed at a plurality of time points. Here, the “time point” does not have a strict meaning such as the time when the examination is performed, and is, for example, an examination date. In addition, the data acquisition function 152 may acquire examination values of not only one type of biological examination but also a plurality of types of biological examinations as the examination values. In this case, each biological examination is performed at a plurality of same time points, but may not be exactly the same examination date.

As such, the data acquisition function 152 acquires examination data (time series data) including examination values of one or a plurality of types of biological examinations performed on the same subject at a plurality of time points.

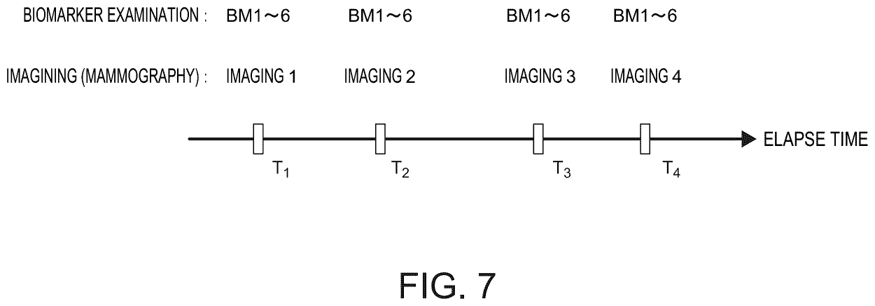

The heat map creation function 153 creates a heat map for grasping a degree of risk (disease risk) for each biomarker based on the examination data. A method of creating the heat map will be described below with reference to FIGS. 7 to 10. It should be noted that a “heat map” is also simply referred to as a “map”.

The display control function 154 controls the output interface 13 to display a GUI or the like. For example, the display control function 154 causes the output interface 13 to display the heat map or the like created by the heat map creation function 153. Note that the display control function 154 may control another information processing apparatus (not illustrated) connected to the medical information processing apparatus 10 via the communication network N to display the heat map or the like.

The display control function 154 acquires, from the operation reception function 151, information regarding which element is selected by the user from among the plurality of elements constituting the heat map. Then, the display control function 154 controls the output interface 13 to display medical-related information related to one or the plurality of selected elements.

The medical-related information is, for example, a graph (time course) of the examination values of the biological examinations corresponding to one or the plurality of selected elements. The medical-related information may be a fluorescence microscopic image or a co-localization Venn diagram of a biological examination corresponding to one or the plurality of selected elements.

Note that the medical-related information may be a medical image (a mammography image, an ultrasound image, a CT image, an MRI image, or the like) captured at the same time point as the biological examination of the selected element. In this case, the display control function 154 may cause the output interface 13 to display an enlarged image of a suspicious site (such as a site suspected of cancer) in the medical image and/or an image finding based on the medical image.

The relevant BM extraction function 155 extracts the type of biological examination (biomarker) highly relevant to (the therapeutic effect of) the subtype or drug specified by the user. For the extraction processing, a database in which highly relevant biomarkers are stored in association with each subtype (or drug) may be used, or a learned model obtained by machine learning of a relationship of therebetween may be used. Note that the relevant BM extraction function 155 can also extract a highly relevant subtype or drug from the types of biomarkers by using the database and the learned model by switching input and output.

Note that the relevant BM extraction function 155 may extract one or a plurality of biomarkers highly relevant to a disease subgroup to which the patient corresponds among a plurality of types of biological examinations conducted to the patient based on the medical image and/or the patient information.

When a highly relevant biological examination type is extracted as described above, the heat map creation function 153 rearranges a plurality of time-series element groups so that time-series element groups corresponding to the extracted biological examination are grouped. As a result, it is possible to display a heat map that bundles the examination results of highly related biological examinations. Note that the display control function 154 may control the output interface 13 to display the time-series element group corresponding to the extracted biological examination on an upper side of the heat map, or to display only the time-series element group corresponding to the extracted biological examination.

Note that the subtype is, for example, a subtype of breast cancer, and is obtained by classifying breast cancer according to characteristics of genes of breast cancer cells. The subtype of breast cancer includes ductal carcinoma, solid ductal carcinoma, hard carcinoma, luminal A cancer, HER2-positive cancer, and the like. The drug is, for example, an anticancer agent for cancer.

### <Medical Information Storage Apparatus>

A medical information storage apparatus 20 according to a first embodiment will be described in detail with reference to FIG. 3.

As illustrated in FIG. 3, the medical information storage apparatus 20 includes a memory 21, a processing circuit 22, and a communication interface 23. Hereinafter, details of each configuration will be described.

The memory 21 is connected to the processing circuit 22 and stores various types of information used in the processing circuit 22. In addition, the memory 21 stores a patient information DB 211, a biomarker DB 212, a medical image DB 213, and a finding information DB 214.

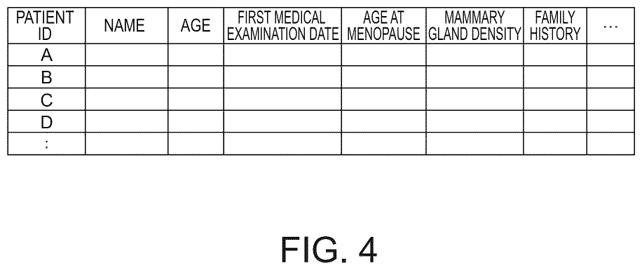

The patient information DB 211 is a database that stores information on a patient. FIG. 4 illustrates an example of the patient information DB 211. In this example, the record of each patient stores information such as patient ID, name, age, first medical examination date, age at menopause, mammary gland density, and family history (medical history of a relative's disease).

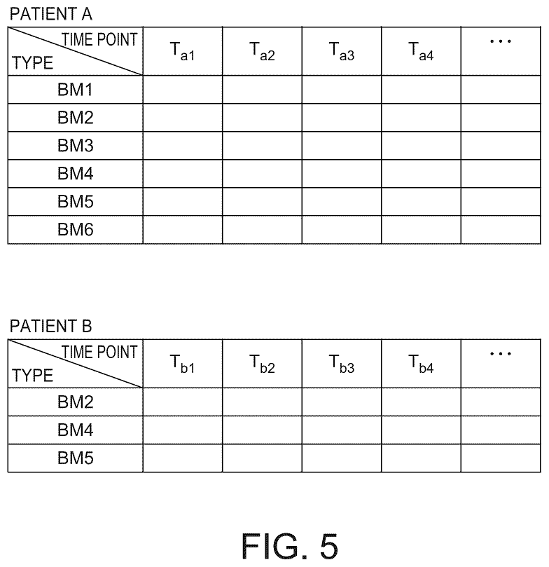

The biomarker DB 212 is a database that stores examination values of biomarker examinations. FIG. 5 illustrates an example of the biomarker DB 212. In this example, the examination value at each examination time point is stored for each patient. In a case of a patient A, examination values of examinations performed at time points Tₐ₁, Tₐ₂, Tₐ₃, Tₐ₄, . . . , and the like are stored for six types of biomarkers BM1 to BM6. Similarly, for patient B, examination values of examinations performed at time points T_{b1}, T_{b2}, T_{b3}, T_{b4}, . . . , and the like are stored for three types of biomarkers BM2, BM4, and BM5. Note that the examination time points of the patient A and the patient B may be different from each other.

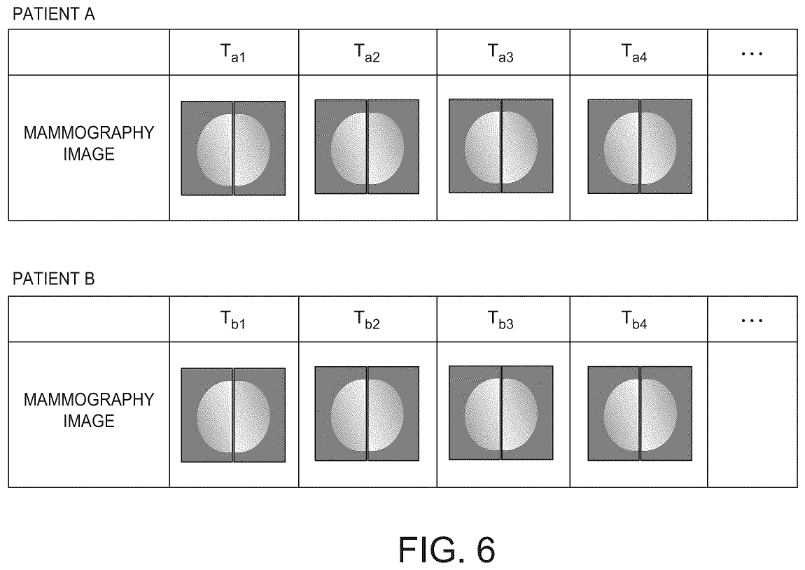

The medical image DB 213 is a database that stores medical images captured by the medical imaging apparatus 30 such as mammography. FIG. 6 illustrates an example of the medical image DB 213. In this example, the medical image (mammography image) at each examination (imaging) time point is stored for each patient.

Note that the patient information DB 211, the biomarker DB 212, and the medical image DB 213 are not limited to be stored in one medical information storage apparatus 20, and may be stored in separate apparatuses. For example, the patient information DB 211 may be stored in a storage apparatus of an electronic health record system connected to the medical information processing apparatus 10 via the communication network N. In addition, the medical image DB 213 may be stored in an apparatus in a picture archiving and communication system (PACS) connected to the medical information processing apparatus 10 via the communication network N.

As can be seen from FIGS. 5 and 6, for each patient, the time points of performing the biological examination and imaging the medical image are the same. However, as described above, the “time point” does not have a strict meaning such as the time when the examination or the imaging is performed, and is, for example, a day when the examination or the imaging is performed. Note that examination and imaging performed in the same period (week, month, and the like) may be treated as being performed at the same time point.

FIG. 7 illustrates an example of the order of biological examination (biomarker examination) and imaging (mammography). In this case, examination of the biomarkers BM1 to BM6 and mammography imaging are performed at time points T₁, T₂, T₃, and T₄. The examination values of the biomarker examinations performed at each time point are stored in the biomarker DB 212. In addition, the mammography image captured at each time point is stored in the medical image DB 213. Examination values of biomarker examinations and mammography images can be associated via examination time points. Note that the time points T₁, T₂, T₃, and T₄ may be at equal intervals or at unequal intervals.

The finding information DB 214 is a database that stores information on a suspicious site (for example, a site suspected of cancer), an image finding, and the like obtained by inputting a medical image to a learned model obtained by machine learning. The learned model for obtaining the information is arranged in a medical learning apparatus (not illustrated) connected to the medical information processing apparatus 10 via the communication network N. For example, when a new medical image is stored in the medical image DB 213, the medical image is transmitted to the medical learning apparatus, and thereafter, the medical information processing apparatus 10 receives an output result of the learned model and stores the output result in the finding information DB 214. Note that the medical information processing apparatus 10 may have a learned model and obtain a suspicious site, an image finding, or the like by itself.

### <Method of Creating Heat Map>

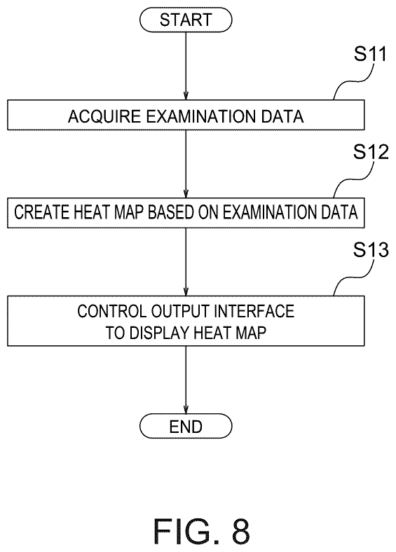

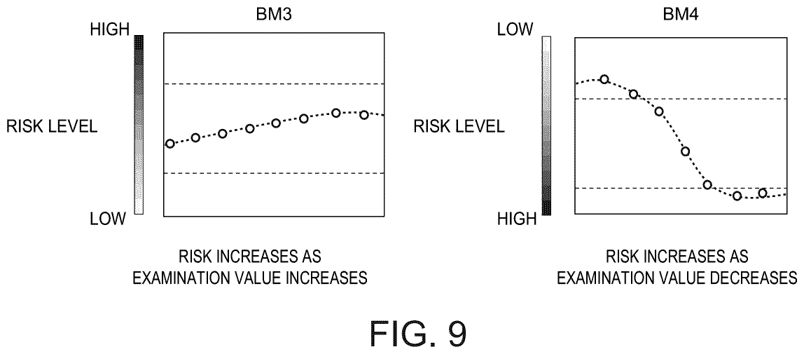

Here, a method of creating and displaying a heat map according to an embodiment will be described with reference to FIGS. 8 to 10. Note that the vertical axis of each graph in FIG. 9 represents an examination value, and the horizontal axis represents a time point.
Step S11: The data acquisition function 152 acquires examination data (time series data of examination values of a biological examination). Specifically, the data acquisition function 152 acquires examination data of a patient designated by the user of the medical information processing apparatus 10 from the medical information storage apparatus 20 (biomarker DB 212).
Step S12: The heat map creation function 153 creates a heat map based on the inspection data acquired in Step S11. First, the heat map creation function 153 converts the examination value of the biological examination into a color corresponding to the degree of risk (risk level). For example, as the risk level increases, the examination value at each time point is converted so as to form a gradation from black to white. The color is not particularly limited, and for example, conversion may be performed so as to form a gradation from red to green. The conversion from the examination value to the color may be performed by, for example, standardizing the examination value to a common risk level (such as 0 to 1) among a plurality of biological examinations, and then converting the standardized value to a predetermined color corresponding to the standardized risk level.

Note that, in a case of monochrome display, for example, the heat map creation function 153 may convert the examination value so as to form a gradation from black to white as the risk level decreases. A relationship between a magnitude of an examination value and a degree of a risk level varies depending on the type of biomarker. For example, as illustrated in FIG. 9, for the biomarker BM3, the risk increases as the examination value increase, and for the biomarker BM4, the risk increases as the examination value decreases.

Next, the heat map creation function 153 creates a time-series element group by arranging the elements having the converted colors along a lateral direction (first direction) in the order of the examination time points. In the present embodiment, the elements are represented by square dots. Note that the shape of the element is not limited to a square.

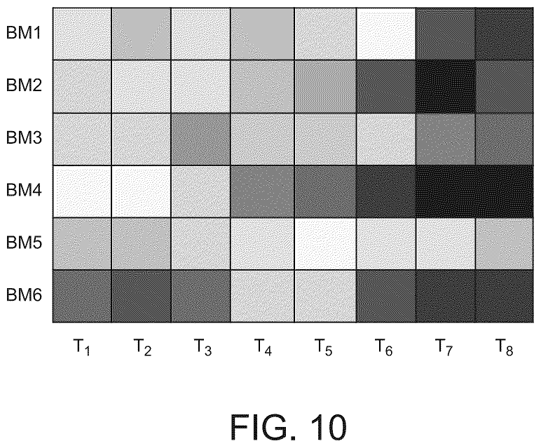

In a case where a plurality of types of biomarker examinations are performed, the time-series element group is created for each type of biomarker. Thereafter, the heat map creation function 153 arranges the time-series element groups of each biomarker examination along a longitudinal direction (second direction). In this way, a heat map is created.
Step S13: The display control function 154 controls the output interface 13 to display the heat map created in Step S12. FIG. 10 illustrates an example of the displayed heat map. In this heat map, each element of the heat map has a color corresponding to the risk level indicated by the biomarker. Therefore, a user such as a doctor does not need to consider the characteristics of each biomarker, and can intuitively grasp the risk level indicated by each biomarker.

The heat map is a heat map of one row in a case where there is one type of biomarker examination. In addition, even in a case where a plurality of types of biomarker examinations are performed, in a case where there is only one examination time point, the heat map is one row. Note that rows and columns may be interchanged in the display of the heat map. That is, elements having the converted colors may be arranged in the longitudinal direction to form a time-series element group, and a plurality of time-series element groups corresponding to the plurality of biomarker examinations may be arranged in the lateral direction to form a heat map.

In addition, the heat map creation function 153 may rearrange the plurality of time-series element groups based on a sorting index. The sorting index can be designated by the user. For example, in a case where the “risk order” is designated as the sorting index, the heat map creation function 153 creates a heat map in which the time-series element groups are sorted in the risk order (for example, the order of the biomarkers with high risk levels on the latest examination date) based on the examination value at the latest examination time point. Note that the heat map creation function 153 may rearrange the plurality of time-series element groups in order of risk based on the examination value at the time point designated by the user.

In the above description, the heat map creation function 153 converts the examination value of the biological examination into a color corresponding to the risk level, but the examination values may be converted into visual representations other than colors. For example, the heat map creation function 153 may convert the examination value into a mark, a letter such as an alphabet, or a pattern according to the risk level. When converting the examination values into marks, the heat map creation function 153 may convert the examination values into “◯”, “Δ”, and “x” as the risk level increases. When converting the examination values into letters, the heat map creation function 153 may convert the examination values into “A”, “B”, “C”, . . . as the risk level increases.

### Screen Example

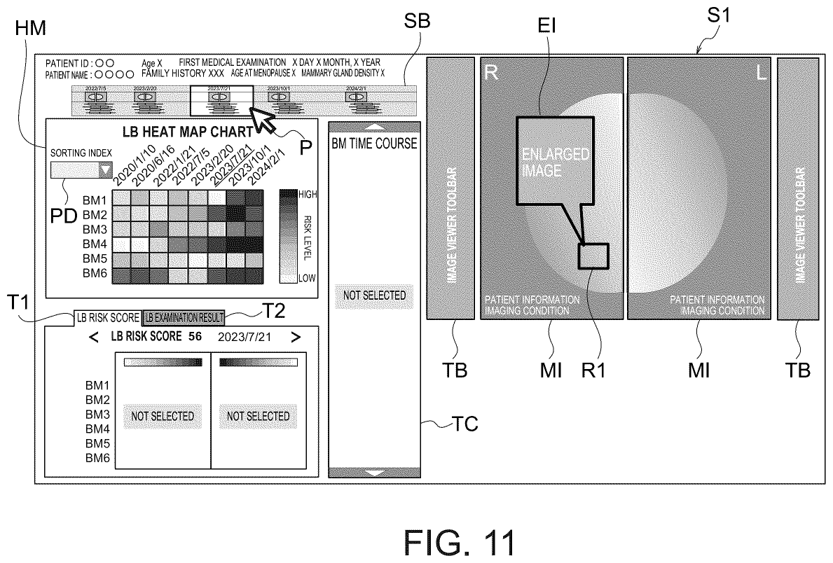

Screen examples displayed on the display unit of the output interface 13 of the medical information processing apparatus 10 will be described with reference to FIGS. 11 to 14.

A screen S1 of FIG. 11 illustrates a screen example when the user of the medical information processing apparatus 10 selects a patient to be diagnosed from a patient list (not illustrated) and selects an examination date (here, Jul. 21, 2023) of the selected patient. In the upper left of the screen, patient information (patient name, age, or the like) of the selected patient is displayed.

On the screen S1, the user can select the examination date of the biological examination from a selection band SB by a pointer P Note that, as illustrated in FIG. 11, types of the medical image and the biological examination may be reduced and displayed on the selection band SB. In addition, an interval of a date display in the selection band SB may be changed and displayed according to the interval of the examination date.

In a heat map display area HM, a heat map including the examination value of the selected examination date is displayed. In the present embodiment, a pull-down menu PD for selecting a sorting index is arranged in the heat map display area HM. In addition, the examination date is displayed in association with the element of the heat map. Among them, the examination date selected in the selection band SB is underlined. As such, the examination date selected by the selection band SB may be displayed in a display mode (different colors, fonts, and the like) different from other examination dates.

In a medical image display area MI, a medical image captured on the examination date selected in the selection band SB is displayed. In the screen example of FIG. 11, the mammography image of the right breast of the patient is displayed in the left medical image display area MI, and the mammography image of the left breast of the patient is displayed in the right medical image display area MI. Note that patient information, imaging conditions, and the like may be displayed in the medical image display area MI. Various tools of an image viewer for viewing the medical image are displayed on a toolbar TB.

As illustrated in FIG. 11, in the medical image of the medical image display area MI, based on the finding information DB 214, a site suspected of cancer is displayed as an attention area R1, and an enlarged image EI of the attention area R1 is further displayed. Note that the enlarged image EI may be displayed by the user by selecting the attention area R1 with the pointer P or the like.

In displaying a medical image, first, the data acquisition function 152 acquires a medical image captured on the examination date selected by the selection band SB from the medical image DB 213 of the medical information storage apparatus 20. Then, the display control function 154 controls the output interface 13 so that the acquired medical image is displayed in the medical image display area MI. Note that the data acquisition function 152 may acquire all the medical images of the patient before the examination date is selected at a timing when the patient is selected.

Note that when the user selects an element of the heat map on the screen S1, the medical image displayed in the medical image display area MI may be changed accordingly. That is, the medical image of the examination date corresponding to the element selected in the heat map may be displayed in the medical image display area MI. In this case, the data acquisition function 152 acquires a medical image captured on the examination date corresponding to the element selected in the heat map from the medical image DB 213 of the medical information storage apparatus 20, and the display control function 154 controls the output interface 13 so that the acquired medical image is displayed in the medical image display area MI. Note that, in a case where a plurality of elements are selected, a medical image may be displayed based on a predetermined rule. For example, a medical image corresponding to an element having the latest examination date among the plurality of selected elements may be displayed.

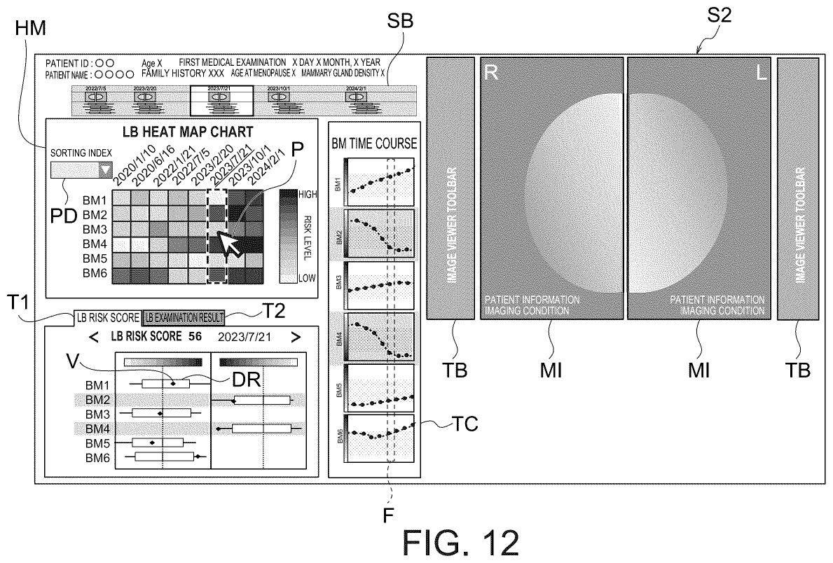

On the screen S1 of FIG. 11, the user can select an element of the heat map by the pointer P. FIG. 12 illustrates a screen S2 in a case where BM1 to BM6 on Jul. 21, 2023 are selected. As illustrated in FIG. 12, a risk score (LB risk score) calculated based on the examination result of the liquid biopsy on the selected examination date is displayed on a tab T1. This risk score is, for example, a value of 0 or more and 100 or less. The higher the risk, the higher the value. In addition, in the tab T1, an examination value V is displayed together with a distribution range (normal range) DR in a healthy human for each biomarker. As a result, it is possible to easily grasp whether or not the examination value is within the normal range for each biomarker.

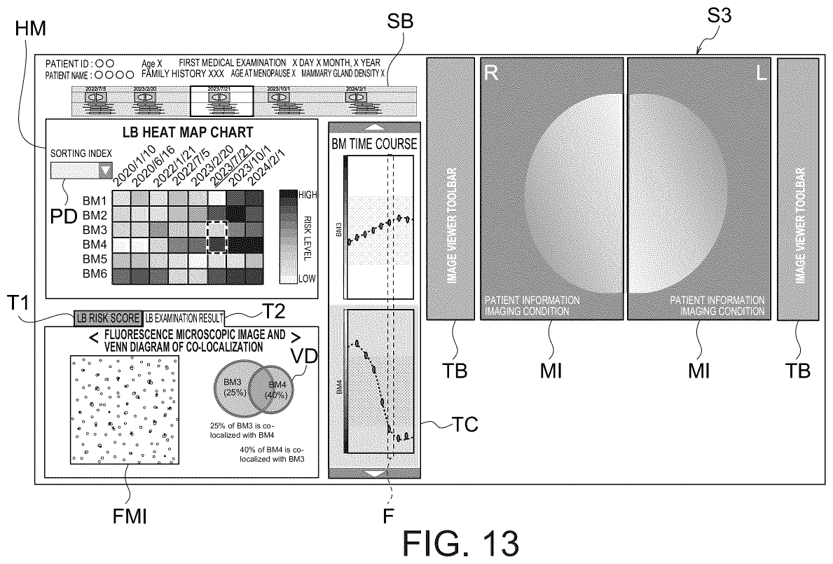

As illustrated in FIG. 12, a graph of the examination results of the biomarkers (here, BM1 to BM6) corresponding to the elements selected in the heat map is displayed in a time course display area TC. In FIG. 13, since only two biomarkers BM3 and BM4 are selected, a graph of the examination results of BM3 and BM4 is displayed in the time course display area TC. In the present embodiment, the examination value of the examination date corresponding to the element selected in the heat map is surrounded by a frame F. Note that, although not limited thereto, the examination value may be displayed in a distinguishable manner by displaying the examination value in a display mode such as a color different from the examination value of the other examination dates.

As shown in a screen S3 of FIG. 13, an examination result (raw data) such as a fluorescence microscopic image FMI is displayed on a tab T2. In the fluorescence microscopic image FMI, the examination result of the biological examination (biomarker) corresponding to the element selected in the heat map may be displayed. In the example of FIG. 13, since BM3 and BM4 on Jul. 21, 2023 are selected, a fluorescence microscopic image of BM3 and BM4 on the examination date are displayed in a superimposed manner. Note that a Venn diagram VD of co-localization between the biomarkers selected in the heat map may be displayed.

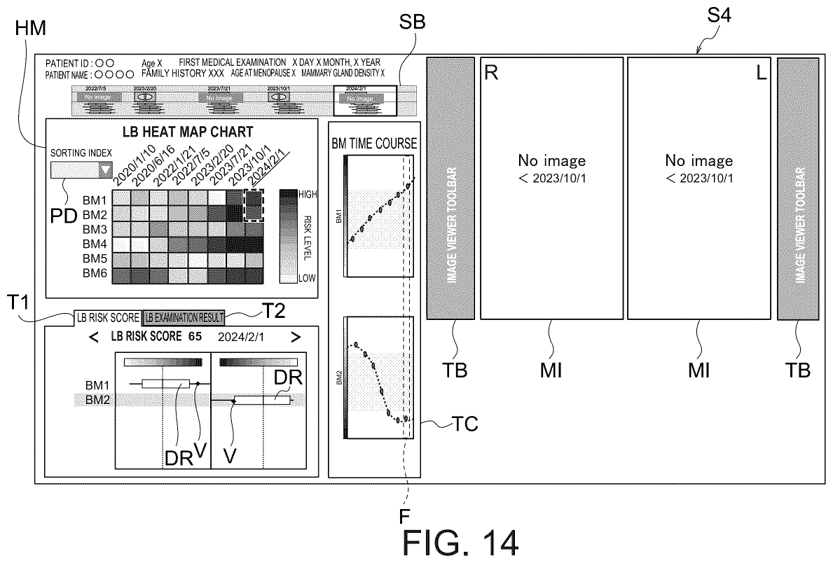

Note that, as shown in a screen S4 of FIG. 14, a display in which it is possible to grasp whether or not a medical image corresponding to the examination date of the biological examination exists in the selection band SB may be displayed. On the screen S4, the examination date on which no medical image exists is displayed as “No image”. As a result, the user can easily select the examination date on which both the examination result of the biological examination and the medical image exist. Note that the display mode is not limited thereto, and for example, in the selection band SB of the screen S1 illustrated in FIG. 11, a reduced image, an icon, or the like indicating a medical image may not be displayed on the examination date on which no medical image exists. In addition, the display may be performed in the heat map. For example, the display mode of the examination date displayed in the heat map may be changed depending on the presence or absence of the medical image. For example, the examination date on which the medical image exists may be displayed in a different color or font, or may be underlined.

Next, a case where the sorting index is selected from the pull-down menu PD will be described. In the present embodiment, the user can select “risk order”, “by subtype”, or “by drug” as the sorting index.

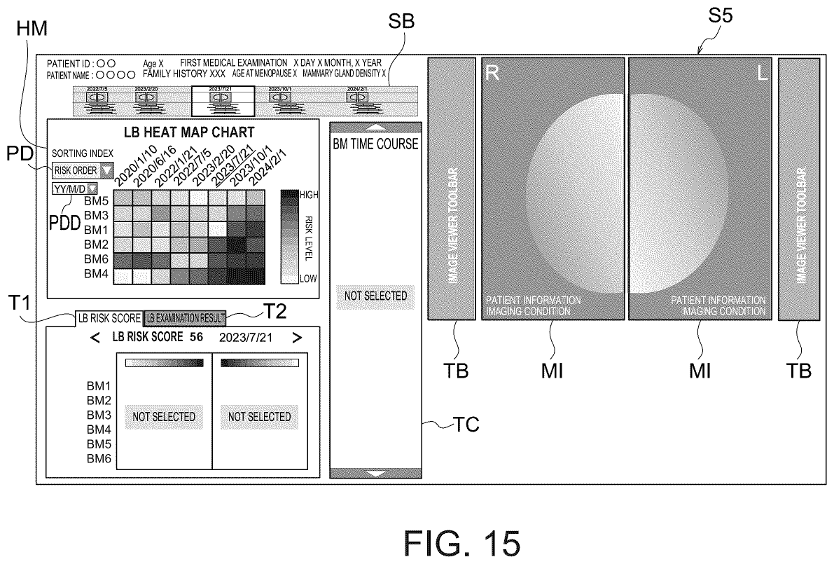

FIG. 15 illustrates a screen S5 in a case where “risk order” is selected from the pull-down menu PD. In the heat map display area HM, the heat map in which the time-series element groups of the biomarkers BM1 to BM6 are arranged from the top to the bottom in the ascending order of the risk levels on the latest examination date (here, Feb. 1, 2024) is displayed. That is, in this example, the biomarker BM4 indicates the highest risk level and the biomarker BM5 indicates the lowest risk level on the latest examination date. Note that the time-series element groups may be arranged from the top to the bottom in the descending order of the risk level.

A pull-down menu PDD is for designating an examination date serving as a sorting reference. In the present embodiment, when no designation is made, sorting of the time-series element groups is performed in the order of risk on the latest examination date as described above. On the other hand, in a case where the user inputs the examination date from the pull-down menu PDD, the heat map in which the time-series element groups are sorted in the order of risk on the examination date is displayed.

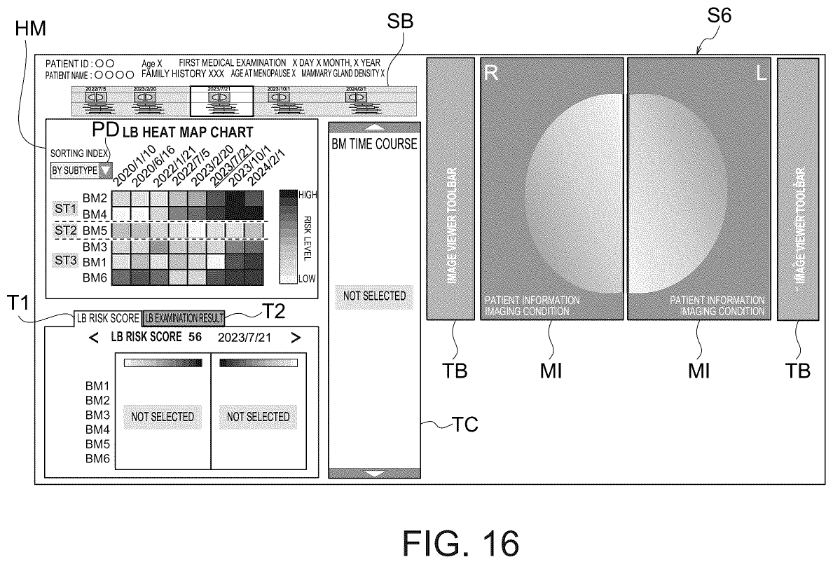

FIG. 16 illustrates a screen S6 in a case where “by subtype” is selected from the pull-down menu PD. In the heat map display area HM, a heat map including the time-series element groups of the biomarkers BM1 to BM6 sorted by subtype (here, ST1, ST2, and ST3) is displayed. That is, the heat map sorted by a group of the biomarkers BM2 and BM4 related to the subtype ST1, a group of the biomarkers BM5 related to the subtype ST2, and a group of the biomarkers BM3, BM1, and BM6 related to the subtype ST3 is displayed.

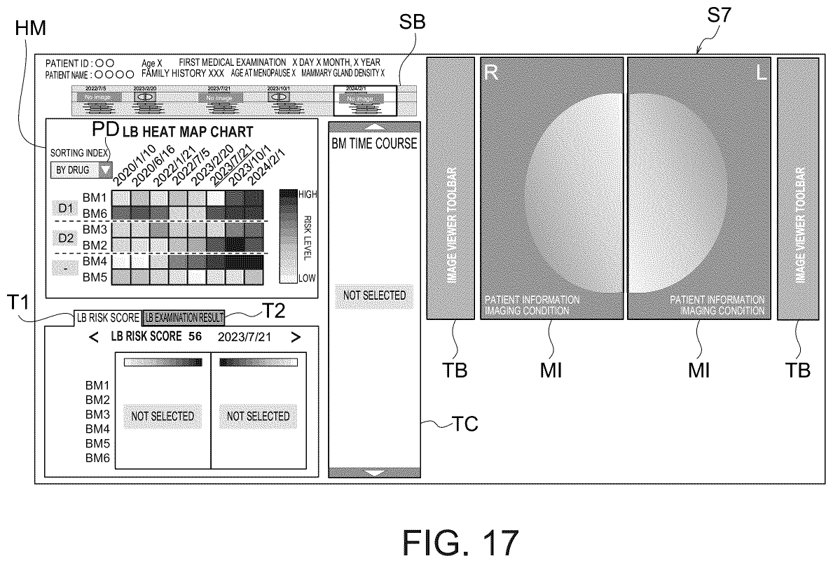

FIG. 17 illustrates a screen S7 in a case where “by drug” is selected from the pull-down menu PD. In the heat map display area HM, a heat map including the time-series element groups of the biomarkers BM1 to BM6 sorted by drug (here, D1, D2, and the like) is displayed. That is, the heat map sorted by a group of the biomarkers BM1 and BM6 related to the drug D1, a group of the biomarkers BM3 and BM2 related to the drug D2, and a group of the biomarkers BM4 and BM5 related to other drugs (drugs not designated) is displayed. Note that, in FIG. 17, the time-series element group of the biomarkers related to the non-designated drug is also displayed, but the present invention is not limited thereto, and only the time-series element group of the biomarkers of the designated drug may be displayed.

Processing contents in the medical information processing apparatus 10 in a case where the sorting index is selected will be described. The operation reception function 151 receives a user's operation on the pull-down menu PD and grasps the selected sorting index. Thereafter, the heat map creation function 153 rearranges the time-series element group of BM1 to BM6 based on the specified sorting index. In a case where the sorting index is “risk order”, the heat map creation function 153 creates a heat map sorted in the risk order by rearranging the time-series element groups of BM1 to BM6 in the order from a biomarker with a low risk level to a biomarker with a high risk level (or in the reverse order) on the latest examination date (or the specified examination date). Thereafter, the display control function 154 controls the output interface 13 to display the sorted heat map.

In a case where the sorting index is “by subtype”, a subtype highly relevant to each biomarker is extracted using the relevant BM extraction function 155, and the biomarkers are grouped according to the extracted subtype. The heat map creation function 153 rearranges the time-series element groups for each of the grouped biomarkers. In a case where the sorting index is “by drug”, a drug highly relevant to each biomarker is extracted using the relevant BM extraction function 155, and the biomarkers are grouped according to the extracted drug. The heat map creation function 153 rearranges the time-series element groups for each of the grouped biomarkers.

As described above, according to the present embodiment, a heat map including a time-series element group in which a plurality of elements corresponding to the biological examinations performed at a plurality of time points and having a color according to a degree of risk are arranged in a time-series order along a first direction is created and displayed. As a result, a user such as a doctor can visually and easily grasp the risk level indicated by the examination result of the biological examination at the plurality of time points.

In addition, according to the present embodiment, in a case where the examination data includes a plurality of types of biological examinations, a heat map in which a plurality of time-series element groups corresponding to the plurality of types of biological examinations are arranged along a second direction different from the first direction is created and displayed. As a result, a user such as a doctor can easily grasp and compare the risk levels indicated by the examination results of the plurality of types of biological examinations. In addition, the examination data can be displayed on the screen in a space-saving manner by the heat map display.

Furthermore, in the present embodiment, medical-related information (medical image and the like) related to one or a plurality of elements selected by a user such as a doctor among the plurality of elements constituting the heat map is displayed together with the heat map. As a result, it is possible to refer to the selected element (the type of the biological examination or the examination time point) and information related thereto at the same time, and thus, it is possible to support comprehensive diagnosis. As a result, the diagnosis accuracy can be improved. That is, even when it is found that there is a possibility of a disease such as cancer by a biomarker examination by a kit biopsy or the like, it is not possible to specify the site. On the other hand, according to the present embodiment, the comprehensive diagnosis can be supported by presenting the heat map that displays the result of the biomarker examination in an easy-to-understand manner and the medical image captured at the same time point as the biomarker examination to the user such as a doctor. Furthermore, the support effect can be enhanced by presenting the suspicious site and the image finding.

Furthermore, in the present embodiment, a heat map in which the time-series element groups corresponding to the respective biological examinations are rearranged is created and displayed based on a sorting index such as risk order, subtype, or drug. In addition, biological examinations highly relevant to the designated subtype or drug are extracted, and a heat map in which time-series element groups corresponding to the extracted biological examinations are rearranged to be grouped is creased and displayed. By referring to the heat map in which the time-series element groups are rearranged in this manner, the user such as a doctor can improve the diagnosis accuracy.

### Modification of First Embodiment

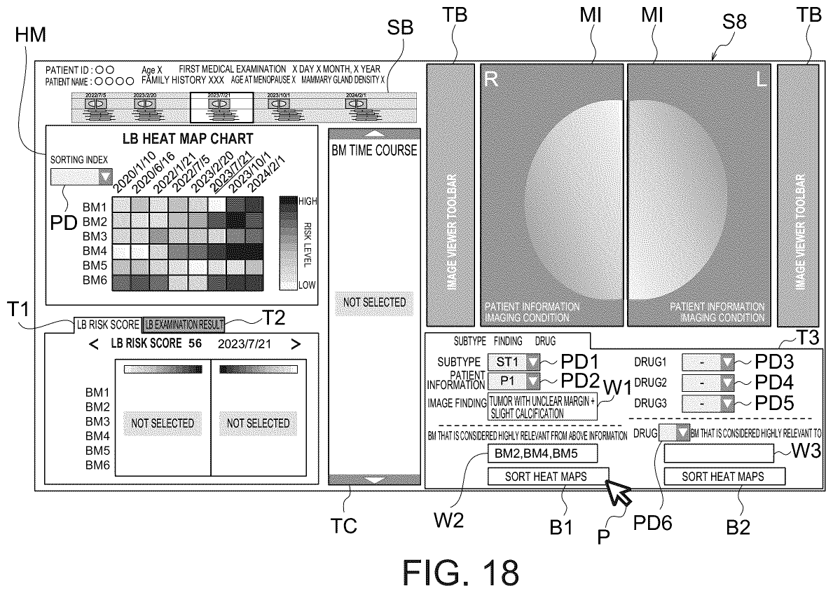

A modification of the first embodiment will be described with reference to FIGS. 18 to 21.

In the present modification, a new tab T3 (“subtype finding drug”) is arranged on a screen, and a user selects a subtype, a drug, or the like from a pull-down menu. A heat map in which biomarkers highly relevant to the selected subtype or drug are grouped is displayed.

On a screen S8 illustrated in FIG. 18, a subtype (ST1, ST2, ST3, or the like) can be selected from a pull-down menu PD1, and patient information can be selected from a pull-down menu PD2. The patient information is, for example, a family history, and is selected from hypertension, diabetes, heart disease, and the like. In this screen example, ST1 is selected as the subtype from the pull-down menu PD1, and P1 (for example, diabetes) is selected as the patient information from the pull-down menu PD2. In a message window W1, the image finding (“tumor with unclear margin+slight calcification”) read from a finding information DB 214 is displayed.

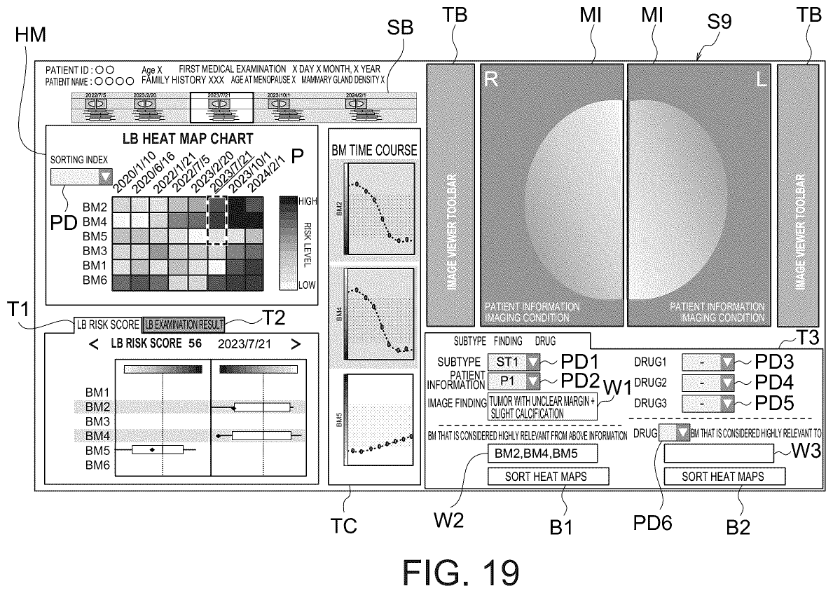

When the subtype and patient information are selected as described above, BM2, BM4, and BM5 are displayed in a message window W2 as biomarkers highly relevant to the subtype ST1. Here, when a user presses a button B1, BM2, BM4, and BM5 are grouped and displayed in the upper order of the heat map as in a screen S9 illustrated in FIG. 19. In addition, examination values and distribution ranges of BM2, BM4, and BM5 are displayed on a tab T1, and graphs of BM2, BM4, and BM5 are displayed in a time course display area TC.

Note that, on the screen S9, a color tone or the like of the highly relevant biomarker element may be changed so that the highly relevant biomarker can be easily distinguished from another biomarker element. For example, the element of the time-series element group corresponding to the highly relevant biomarker may be displayed to have a gradation from yellow to blue as a risk level change from a high risk to a low risk.

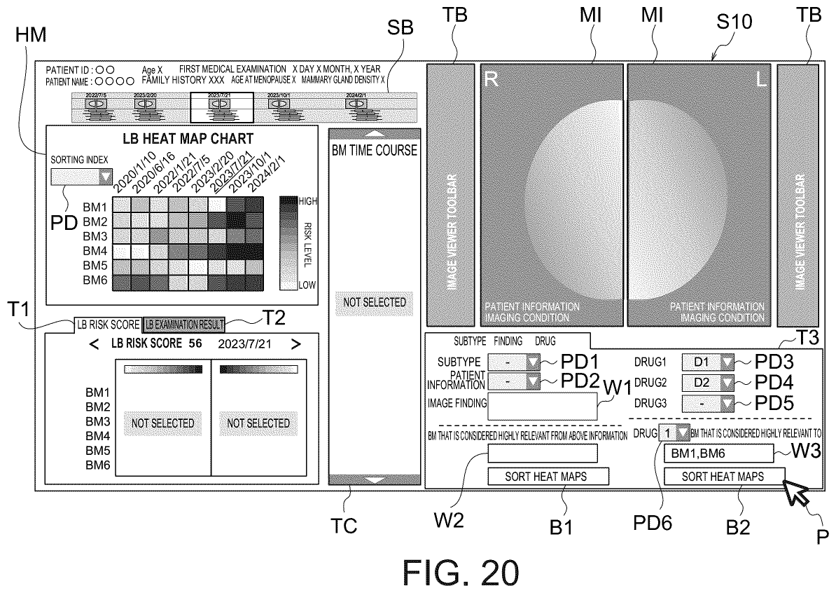

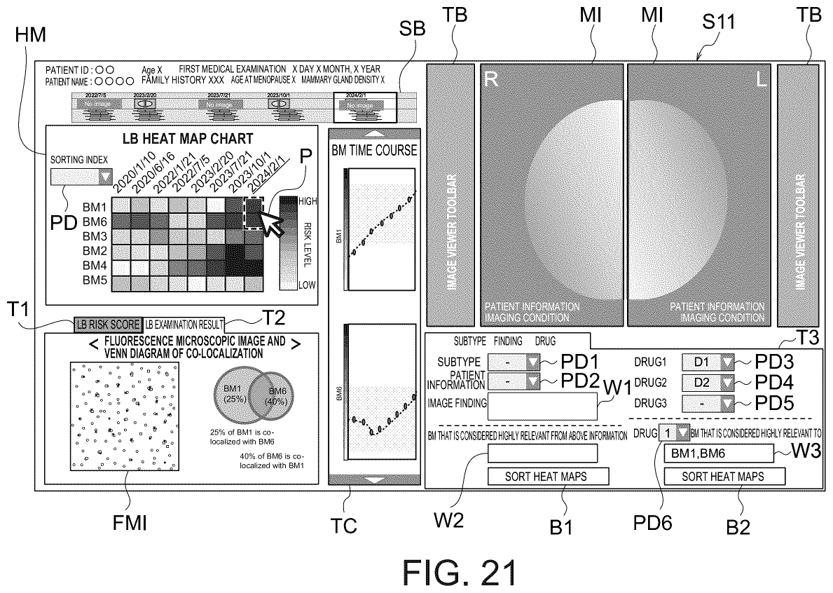

On a screen S10 illustrated in FIG. 20, up to three types of drugs can be selected from pull-down menus PD3 to PD5. One of the drug 1 to the drug 3 can be selected from a pull-down menu PD6. Here, “D1” is selected as the drug 1, “D2” is selected as the drug 2, and the drug 1 is selected from the pull-down menu PD6. When the drug is selected, BM1 and BM6 are displayed in a message window W3 as biomarkers highly relevant to the drug D1. Here, when a user presses a button B2, BM1 and BM6 are grouped and displayed in the upper order of the heat map as in a screen S11 illustrated in FIG. 21. In addition, examination values and distribution ranges of BM1 and BM6 are displayed on the tab T1, and graphs of BM1 and BM6 are displayed in the time course display area TC. In addition, a fluorescence microscopic image of BM1 and BM6 and a Venn diagram of co-localization of BM1 and BM6 are displayed in a tab T2.

Note that, on the screens S9 and S11, only a time-series element group corresponding to a highly relevant biomarker may be displayed. In addition, a highly relevant biomarker may be automatically selected, and medical-related information such as an examination value may be displayed in the tab T1, the tab T2, and the time course display area TC.

### Medical Information Processing Apparatus (Second Embodiment)

A medical information processing apparatus 10A according to a second embodiment will be described in detail. One of the differences between the second embodiment and the first embodiment is that a score calculated based on an examination value of a biomarker is displayed. Hereinafter, the second embodiment will be described focusing on the differences.

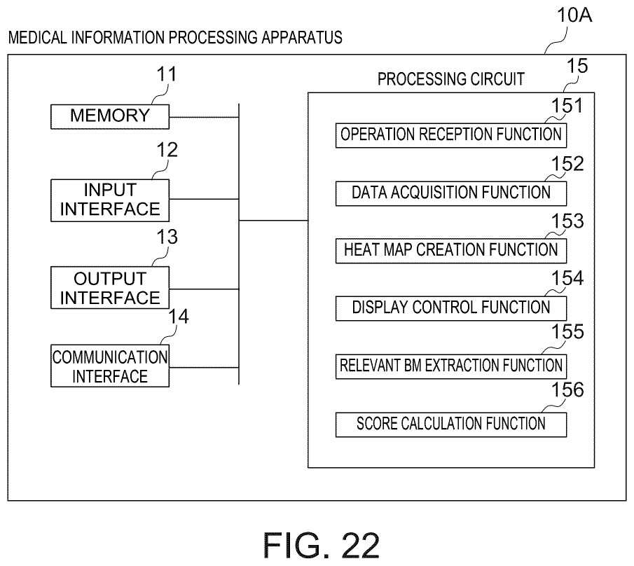

As illustrated in FIG. 22, the medical information processing apparatus 10A includes a memory 11, an input interface 12, an output interface 13, a communication interface 14, and a processing circuit 15. Since the configuration other than the processing circuit 15 is the same as that of the first embodiment, a detailed description thereof will be omitted.

The processing circuit 15 has an operation reception function 151, a data acquisition function 152, a heat map creation function 153, a display control function 154, a relevant BM extraction function 155, and a score calculation function 156. Since the configuration other than the score calculation function 156 is the same as that of the first embodiment, a detailed description thereof will be omitted.

The score calculation function 156 calculates a score based on examination data of a biological examination corresponding to one or a plurality of elements selected by a user among the elements of the heat map. A method of calculating the score is not particularly limited, and for example, a risk level based on the examination value of the biological examination is multiplied by the same or different weighting factor to obtain a sum. Alternatively, a learned model obtained by machine learning a relationship between the examination result of the biological examination and the score may be used. Note that the score calculation function 156 may calculate a score based on the examination values of all the examined biological examinations regardless of the selection of the element of the heat map by the user.

In the present embodiment, the score calculation function 156 calculates a score (integrated score) in consideration of a medical image captured at the same time point as the biological examination in addition to the examination value of the biological examination. That is, the score calculation function 156 calculates an integrated score based on the biological examination and the medical image. The examination value used to calculate the integrated score may be an examination value of a biological examination corresponding to one or a plurality of elements selected by the user, or may be an examination value of all biological examinations on the selected examination date.

Note that the integrated score may be calculated in consideration of information (suspected site, image finding, and the like) in the finding information DB 214 and/or patient information such as a family history. A method of calculating the integrated score is not particularly limited, and for example, the integrated score may be calculated by multiplying each risk level based on the examination value of the biological examination and the medical image by the same or different weighting factors and taking the sum of them. Alternatively, the integrated score may be calculated using a learned model obtained by machine learning in advance.

In addition, in a case where an attention area R1 indicating the suspicious site is displayed in a medical image display area MI as in the screen S1 of FIG. 11, the user may select attention area R1 by clicking the attention area R1 with a pointer P or the like so that the attention area R1 is reflected in the integrated score. For example, the score calculation function 156 may calculate the integrated score in consideration of the finding information in the finding information DB 214.

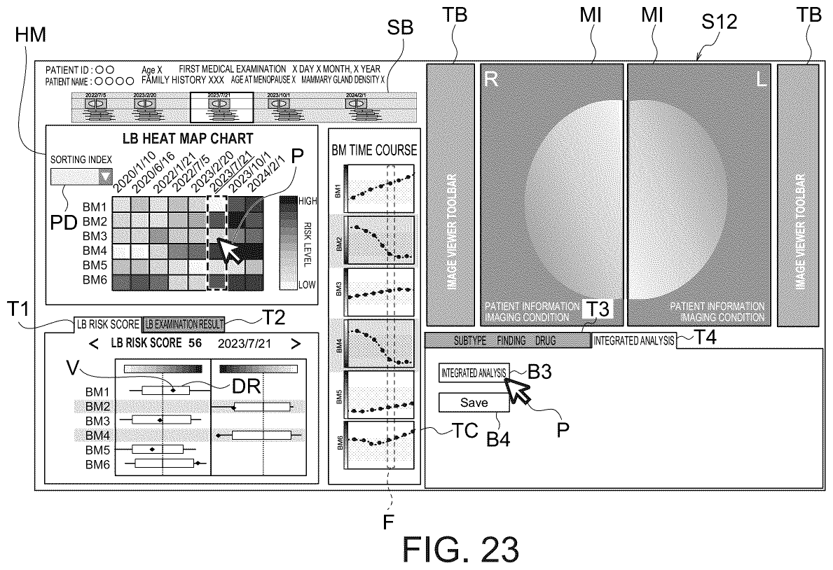

In a screen S12 illustrated in FIG. 23, a tab T4 (“integrated analysis”) is arranged at the lower right of the screen. The tab T4 is provided with an “integrated analysis” button B3 and a “save” button B4. As illustrated, in the heat map, the elements corresponding to the examinations for the biomarkers BM1 to BM6 on the examination date (Jul. 21, 2023) are selected. In this state, when the user presses the button B3 with the pointer P, the score calculation function 156 calculates the integrated score based on the examination values of the biomarkers BM1 to BM6 on the examination date and the medical image on the examination date. The display control function 154 controls an output interface 13 to display the calculated integrated score.

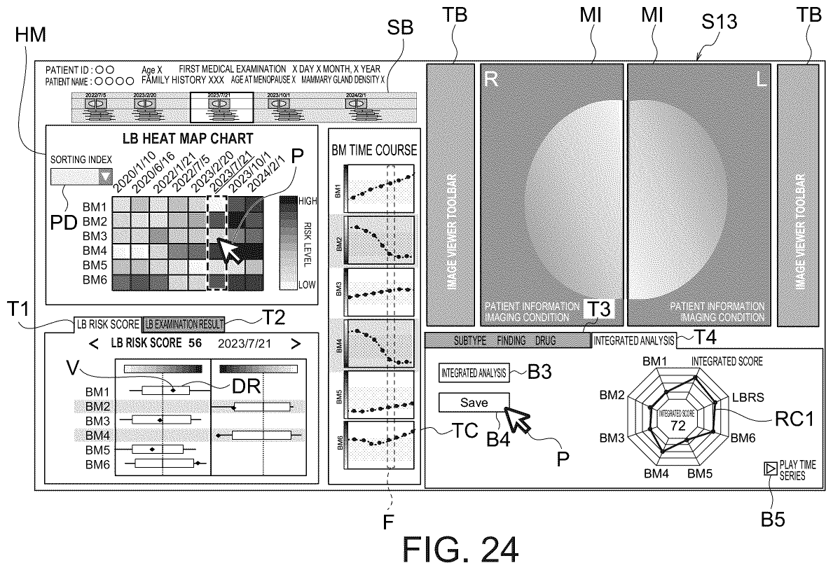

On a screen S13 in FIG. 24, the integrated score (here, “72”) is displayed at the center of a radar chart RC1 in the tab T4. In this screen example, the radar chart RC1 also displays scores and LB risk scores (LBRS) based on the examination values of the respective biological examinations (BM1 to BM6). When the button B4 is pressed on the screen S13, the data of the integrated analysis result such as the radar chart RC1 is stored.

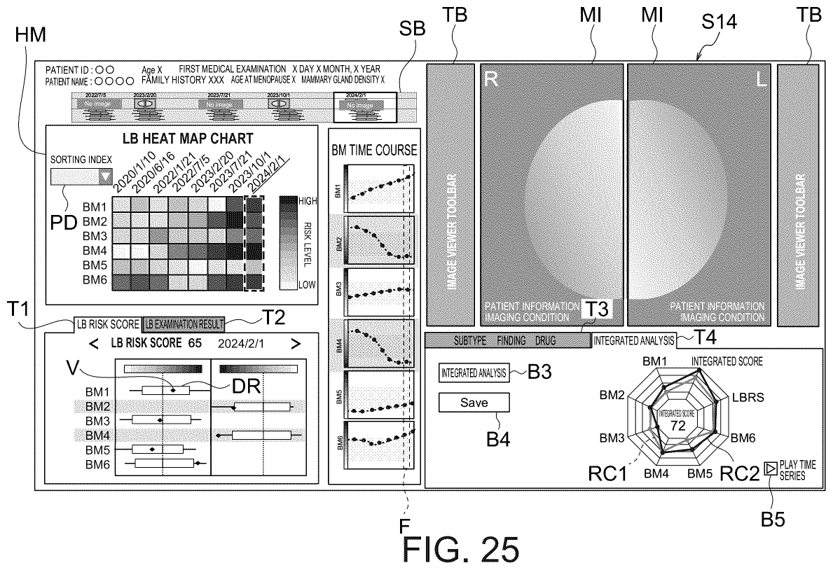

Note that the displayed radar chart may be changed according to the element selected in the heat map of the screen S12. In the screen S14 illustrated in FIG. 25, the elements corresponding to the examinations of the biomarkers BM1 to BM6 on the latest examination date (Feb. 1, 2024) are selected. In this state, when the user presses the button B3, the score calculation function 156 calculates the integrated score based on the examination values of the biomarkers BM1 to BM6 on the examination date and the medical image on the examination date. The display control function 154 controls an output interface 13 to display the calculated integrated score. On the screen S14, a radar chart RC2 corresponding to the latest examination date is overlaid on the radar chart RC1. Note that only the radar chart RC2 may be displayed.

When the radar chart RC2 is displayed, display modes of the radar chart RC1 and the radar chart RC2 may be different from each other. That is, the display forms of the radar charts having different examination dates may be different from each other. For example, as illustrated in FIG. 25, a radar chart with an older examination date is displayed in a lighter color. Alternatively, the line type and color of each radar chart RC1 may be changed. In the present embodiment, when a “time-series reproduction” button B5 is pressed, the radar charts are displayed in the time-series order from the radar chart with the oldest examination date.

As described above, according to the present embodiment, a score calculated based on the examination data of the biological examination corresponding to one or a plurality of elements selected by the user from among the plurality of elements constituting the heat map is displayed. As a result, the user such as a doctor can improve the diagnosis accuracy.

According to the present embodiment, the radar chart created at each designated time point can be superimposed and displayed in a different display mode, or can be sequentially displayed in the time series order. As a result, the risk level and the integrated score along the time series can be easily grasped, and the comprehensive diagnosis can be supported.

Note that the term “processor” used in the above description means, for example, a central processing unit (CPU), a graphics processing unit (GPU), or a circuit such as an application specific integrated circuit (ASIC) or a programmable logic device (for example, a simple programmable logic device (SPLD), a complex programmable logic device (CPLD), or a field programmable gate array (FPGA)). The processor realizes the function by reading and executing the program stored in the memory 11. Note that, instead of storing the program in the memory 11, the program may be directly incorporated in the circuit of the processor. In this case, the processor realizes the function by reading and executing the program incorporated into the circuit. Note that the processor is not limited to a case of being configured as a single processor circuit, and a plurality of independent circuits may be combined to be configured as one processor to realize the function. Furthermore, a plurality of components in FIG. 2 may be integrated into one processor to realize the function.

In addition, the medical information processing method described in FIG. 8 can be realized by executing a medical information processing program prepared in advance on a computer such as a personal computer or a workstation. The medical information processing program can be distributed via a network such as the Internet. The medical information processing program can also be executed by being recorded on a non-transitory computer-readable recording medium such as a hard disk, a flexible disk (FD), a CD-ROM, an MO, or a DVD and being read from the recording medium by the computer.

According to at least one embodiment described above, the risk level indicated by the examination result of the biological examination can be easily grasped.

Although several embodiments have been described above, these embodiments have been presented only as examples, and are not intended to limit the scope of the invention. The novel apparatuses and methods described in the present specification can be implemented in various other forms. In addition, various omissions, substitutions, and changes can be made to the forms of the apparatus and the method described in the present specification without departing from the gist of the invention. The appended claims and their equivalents are intended to include such forms and modifications as fall within the scope and spirit of the invention.

1. A medical information processing apparatus comprising:
processing circuitry configured to
acquire examination data including examination values of biological examinations performed on the same subject at a plurality of time points;
create a map based on the examination data, the map including a time-series element group in which a plurality of elements corresponding to the biological examinations performed at the plurality of time points and having a visual representation according to a degree of risk are arranged in a time-series order along a first direction; and
control a display unit to display the map.

2. The medical information processing apparatus according to claim 1, wherein
the examination data includes examination values of a plurality of types of biological examinations performed at the plurality of time points, and
the processing circuitry is further configured to create a map in which a plurality of the time-series element groups corresponding to the plurality of types of biological examinations are arranged along a second direction different from the first direction.

3. The medical information processing apparatus according to claim 2, wherein the processing circuitry is further configured to
rearrange the plurality of time-series element groups based on a sorting index, and
control the display unit to display a map including the plurality of rearranged time-series element groups.

4. The medical information processing apparatus according to claim 3, wherein the sorting index is risk order, by subtype, or by drug.

5. The medical information processing apparatus according to claim 4, wherein in a case where the sorting index is risk order, the processing circuitry is further configured to rearrange the plurality of time-series element groups in the risk order based on an examination value at the latest examination time point or in the risk order based on an examination value at a designated time point.

6. The medical information processing apparatus according to claim 2, the processing circuitry is further configured to
extract a type of biological examination highly associated with a designated subtype or drug, and
control the display unit to display the extracted type of biological examination.

7. The medical information processing apparatus according to claim 6, wherein the processing circuitry is further configured to
rearrange the plurality of time-series element groups so that time-series element groups corresponding to the extracted biological examination are grouped, and
control the display unit to display a map including the plurality of rearranged time-series element groups.

8. The medical information processing apparatus according to claim 1, wherein the processing circuitry is further configured to control the display unit to display medical-related information related to one or a plurality of elements selected among the plurality of elements constituting the map.

9. The medical information processing apparatus according to claim 8, wherein the medical-related information is a medical image captured at the same time point as the biological examination of the selected element.

10. The medical information processing apparatus according to claim 9, wherein the processing circuitry is further configured to cause the display unit to display an enlarged image of a suspicious area in the medical image and/or an image finding based on the medical image.

11. The medical information processing apparatus according to claim 9, wherein the processing circuitry is further configured to control the display unit to perform display capable of grasping whether or not the medical image exists.

12. The medical information processing apparatus according to claim 8, wherein the medical-related information is a graph of an examination value of a biological examination corresponding to one or the plurality of selected elements.

13. The medical information processing apparatus according to claim 8, wherein the medical-related information is a fluorescence microscopic image or a co-localization Venn diagram of a biological examination corresponding to one or the plurality of selected elements.

14. The medical information processing apparatus according to claim 8, wherein the medical-related information is a score calculated based on examination data of a biological examination corresponding to one or the plurality of selected elements.

15. The medical information processing apparatus according to claim 8, wherein the medical-related information is an integrated score calculated based on examination data of a biological examination corresponding to one or the plurality of selected elements and a medical image captured at the same time point as the biological examination.

16. The medical information processing apparatus according to claim 1, wherein the processing circuitry is further configured to control the display unit to display a medical image captured at a time point selected from the plurality of time points.

17. The medical information processing apparatus according to claim 16, wherein the medical image is a mammography image, an ultrasound image, a CT image, or an MRI image.

18. The medical information processing apparatus according to claim 16, wherein the processing circuitry is further configured to control the display unit to display an enlarged image of a suspicious area in the medical image and/or an image finding based on the medical image.

19. The medical information processing apparatus according to claim 1, wherein the map is a heat map, and the visual representation is color.

20. The medical information processing apparatus according to claim 1, wherein the visual representation is mark or pattern.

21. A medical information processing system comprising:
a medical information storage apparatus that stores examination data including examination values of biological examinations performed on the same subject at a plurality of time points; and
a medical information processing apparatus communicably connected to the medical information storage apparatus,
wherein the medical information processing apparatus comprises processing circuitry configured to
acquire the examination data from the medical information storage apparatus;
create a map based on the examination data, the map including a time-series element group in which a plurality of elements corresponding to the biological examinations performed at the plurality of time points and having a visual representation according to a degree of risk are arranged in a time-series order along a first direction; and
control a display unit to display the map.

22. A medical information processing method comprising:
acquiring examination data including examination values of biological examinations performed on the same subject at a plurality of time points;
creating a map based on the examination data, the map including a time-series element group in which a plurality of elements corresponding to the biological examinations performed at the plurality of time points and having a visual representation according to a degree of risk are arranged in a time-series order along a first direction; and
controlling a display unit to display the map.
